(19) United States
(12) Patent Application Publication
(10) Pub. No.: US 2002/0106346 A1
(43) Pub. Date: Aug. 8, 2002

(54) Immune modulation with polystyrene sulfonate

(76) Inventor: Kumarpal A. Shah, Searington, NY (US)

Correspondence Address: KEUSEY, TUTUNJIAN & BITETTO, P.C., 14 VANDERVENTER AVENUE, SUITE 128, PORT WASHINGTON, NY, 11050, US

(21) Appl. No.: 09/955,803

(22) Filed: Sep. 19, 2001

Related U.S. Application Data
(63) Continuation-in-part of application No. 09/519,229, filed on Mar. 6, 2000, now abandoned.

(51) Int. Cl. A61K 31/74

(52) U.S. Cl. 424/78.27

(57) ABSTRACT

Polystyrene sulfonate (PSS) is a known immune-modulation drug for complement inhibition. Our due diligent review of this drug identifies it as a broad-spectrum immune modulation drug that therapeutically impacts innate and adaptive immune functions. The drug is first of a class of drug that modulates immune responses in accordance to recent advances of immunology. The therapeutic applications with relation to HIV are detailed.

### CROSS REFERENCE TO RELATED APPLICATIONS

This application is a continuation-in-part of my pending patent application, Ser. No. 09/519,229 titled “Method for Immune Switching”, filed on Mar. 6, 2000.

### BACKGROUND OF THE INVENTION

1. Field of the Invention

This invention relates to the administration of Polystyrene sulfonate (PSS) as a broad-spectrum immune modulation drug and has potential for wider therapeutic applications.

2. The Prior Art

Immune related medical problems have caused havoc in medical sciences. The magnitude of challenges posed, for example in HIV/AIDS, is reviewed by Sepkowitz K. A. in “Special Article: AIDS—The first Twenty Years” published in The New England Journal of Medicine, 2001; 244: 1764-1772. The genesis of “Global HIV/AIDS Crisis” is due to a gathering sense of doom in the face of the scale of global epidemic worldwide. 36.1 millions are infected with HIV; an additional 21.8 millions have died; and 13.2 millions children have become “AIDS orphans”. More than 14,000 new infections occur daily—5.3 million in 2000 alone, including 600,000 in children younger than 15 years old. Approximately 70% cases occur in sub-Saharan Africa, where in some regions, seroprevalence of HIV among adults exceeds 25 percent. The Caribbean, Southeast Asia, and Eastern Europe are struggling with substantial rates of new infections. The basic details of this killer HIV is provided by Hardy, Jr. W. D. “The Human Inmune Deficiency Virus”, Medical Clinics NA, 80:1239-1261. In 2000, the Security Council of the United Nations began to address the possibility that AIDS now threatens the world's security. Urgent attempts are now underway to halt the global spreading of HIV.

1. UNAIDS: Currently they are trying to raise $10 Billion Global AIDS fund (New York Times, Jun. 24, 2001, “The UN Looks at AIDS”).

2. Vaccine therapy is still awaited. In 1997, President Bill Clinton challenged scientists to provide an effective vaccine within 10 years. Among the difficulties confronting researchers are viral heterogeneity, uncertainty about how to achieve optimal immunogenecity, the lack of practical animal model, and the ethical dilemmas involved in conducting primary prevention trials in the United States and abroad.

3. International AIDS Activists: They are demanding three Ds: 1.) The first D is for cheaper, better and safer drugs; 2.) The second D is for more dollars for AIDS research; and 3.) The third D for the Debt pardon of the underdeveloped nations so that more funds could be directed to improve infrastructure of medical facilities (Daily News, Jun. 25, 2001) “UN tackles AIDS drug funding”).

4. Pharmaceutical companies: 100s of new drugs are being researched costing multibillion dollars. At stake are the patent laws and availability of these drugs at cost-effective prices to the consumers (a. “Progress against HIV” Nature Biotechnology, S2000; 18: IT 27-29, b. Murphy R. I. “New Antiretroviral drugs in development, AIDS, 2000; S3: 14, S227-234, c. Moore J. P. and Stevenson M. “New Targets for inhibitors of HIV-1 replicators” Nature Reviews/Molecular Cell Biology, 2000; 1: 40-49 and d. Furfine E. S. “The Next Generation of Human inmunedeficiency Virus Protease Inhibitors: Targeting viral resistance, Chapter 4, Proteases as Targets for Therapy, Edited by Klaus von der Helm, Bruce D. Korant and John C. Cheronis, Springer).

5. HIV Experts:

A. World's leading HIV experts such as Anthony Fauci and Cohen O. at National Institute of Health has opined for the need for better, simple and safer drugs for HIV therapy that target HIV genomic structure in a novel way and at multiple points so that the risk of resistance could be reduced (a. Cohen O. J. and Fauci A. “Transmission of drug-resistant strains of HIV-1: unfortunate, but inevitable” The Lancet, 1999; 354:697-698, b. Yerly S. et al “Transmission of antiretroviral-drug resistant HIV-1 variants” Lancet, 1999; 354: 729-733).

B. Another leading expert in immunology, Pinto L. A. from National Institute of Health has commented that in view of the limited therapeutic efficacy of current “HAART” regimes, there is urgent need to combine such therapy with immune based approaches that may improve therapeutic efficacy of present cocktail therapies (Pinto L. A. et al “Short Analytical Review: Immune-Based Approaches for Control of HIV infection and Viral-Induced Immunopathogenesis, in Clinical Immunology, 2000; 97:1-8).

6. Current complexity of HIV management is summed up in the editorial “First Rule of AIDS in Africa: Do No Harm, in The Wall Street Journal, May 2, 2001.

Despite scientific progress and therapeutic advances—cost ineffective medicines have proliferated and the demand-supply gap for the life saving drugs have widened. This is a true international emergency of the 21^{st} century. Leaders around the world are scrambling to find meaningful solutions. Billions of dollars are raised to address the research and development of new drugs and vaccines. Attempts are underway to streamline intellectual property rights so that life saving drugs can be accessed globally at reasonable costs. Attempts are also underway to accelerate and streamline the drug discovery and drug development process so as to benefit consumers without adversely affecting their safety. Cost effective, meaningful, life-saving solutions that can be applied expediently at global level are therefore urgently needed.

Central to the understanding of HIV immunopathogenesis is the working of the human defense system. Evolutionary immunologists date Innate or the natural human defense system to 700 million year or more. It is an ancient anti-microbial defense system. Viruses co-evolved with human existence. Viruses are essentially nucleic acid fragments. They are unable to exist on their own. They must find host and use their replication machinery for its own survival. In order to survive in the human host it must develop strategies to confront and defeat human defenses. A central question in the innate immunity is how they recognize pathogens and how pathogen thwarts the complement system for its survival? Complement system is the ancient innate human defense system against microorganisms including viruses. Considerable advances in the working of the complement System has occurred in the last decade. Pangburn M. K. in “1.2.3 Alternate Pathway: Activation and regulation” in The Complement System, edited By K. Rother, G. O. Till and G. M. Hansch, Springer Verlag Berlin Heidelberg, 1998; 93-115 explains why Alternate complement system fit the description of innate complement system more as opposed to specific pathways of classical and mannose-binding protein. The basic working of the Alternate complement system and its role in the innate host defense system is detailed. According to “enzymatic tick-over hypothesis” there is spontaneous hydrolysis of the thioester in C3 to generate C3b. The role of C3b in selecting self vs. non-self surface is detailed by Walport M. J. “Advances in Immunology: Complement: First of two parts, New England J. of Medicine, 2001; 344: 1058-1066. Activated thioester bond in C3b enables it to bind covalently to hydroxyl groups or nearby carbohydrate and protein acceptor groups. If the acceptor molecule is carbohydrate surface of the bacterium, then the enzyme precursor Factor B binds to the C3b to form C3bB. This is susceptible to cleavage and activation by Factor D. This leads to the formation of a C3 convertase enzyme, C3bBb. This activates complement system to generate C3a to C5a inflammatory intermediate products as well as terminal inflammatory as well as cytotoxic C5b-C9, membrane attack complex. C3bBb stabilizes by the binding of Properdin. This enzyme cleaves more C3 leading to deposition of additional C3b on the bacterium. If the acceptor group is a host cell surface that lacks carbohydrate group, the protective regulatory mechanism come into play. This is illustrated by binding of Factor H as C3bH. This is a cofactor to serine esterase Factor I. The action of Factor I depend upon the normal working of Factor H. Factor I cleaves the C3bH into inactive IC3b. The IC3b no longer can participate in the formation of a C3 convertase enzyme. The carbohydrate environment of the surface on which the C3b is deposited determines the relative affinity of C3b for Factor B. On host cell surfaces bearing polyanions such as sialic acid, Factor H binds with higher affinity than Factor B. On microbial surfaces that lack a polyanionic coating Factor B binds to C3b with a higher affinity than does Factor H. Pangbum M. K., in Immunopharmacology, 2000, 49, 149-157, details the discriminatory power of Factor H to identify self. Factor H is a complement regulatory protein of Alternate complement system. It has a unique structure of twenty short consensus repeat (SCR) domain. Each SCR contain approximately 60 amino acids. Factor H is heavily glycosylated and has a high sialic acid content. Factor H is the key self-recognition protein that discriminates host-like features on microorganisms and generate spectrum of activation rates for the complement system in different microorganisms. A central question in innate immunity is how do its various systems distinguish between potential targets and hosts. Thus recognition of polyanions is not the function of C3b, but a function of Factor H. Factor H uses its 20 SCR domains to search for and interact with many ligands on a given target. Each SCR domain contributes to the recognition pattern, and if these sites work cooperatively in the groups of twos, threes and fours, etc, then by simple combinational math Factor H would have ability to discriminate among over 10⁶ target surfaces. Fearson D. T. and Locksley R. M. in “The Instructive role of Innate Immunity in the Acquired Immune Responses, Science, 1996, 272, 50-54, sets out the fundamental differences in the recognition strategies adapted by innate immunity and adaptive immunity. Innate immunity responses are geared to the recognition of carbohydrate signature in the pathogens. While adaptive immune system is geared to the recognition of peptide fragments that are processed and presented by Antigen presenting cell in the MHC 1 and 11 complexes. In adapting to this system, adaptive immunity has essentially lost the innate ability to identify carbohydrate signature. The key circulating or humeral proteins of the Alternate complement system are Factor D and Factor H that regulate the subsequent fate of C3b and its fragmentations into active and inactive particles. How HIV circumvents the working of the complement system and fools it to gain entry inside cells to facilitate its own survival? Stoiber et al explains this in “The supportive role of complement in HIV pathogenesis” in Immunological Reviews 2001; 180: 168-176. HIV confronts complement system and activate its functioning. However, it also binds Factor H and masks its identity as Self. The crucial role played by Factor H in protecting HIV is evident if HIV is incubated in Factor H depleted sera. This results in 80% complement-dependant virolysis in the presence of HIV specific Abs. C3b fragments are utilized by HIV in the human host to gain entry inside CD 4 cells. Therapeutic options such as blocking Ab against CR2 and the use of Pep A-Factor H derived peptide to thwart immune evasion of HIV were suggested. How Factor H is pirated by pathogens for its own survival advantages? This is detailed by Panguin M. K. 1998 & 2000 as well by German and Finland scientists like Zipfel P. F. et al in “Mini Review: Factor H and disease: a complement regulator affects vital body functions, in Molecular Immunology, 1999; 36: 241-248. Factor H is pirated or its function is subverted by a number of bacteria such as Neiseria Gonococci, Streptococcus Pyogenes, virus such as HIV, Parasites such as Trypanosoma Cruzi. Certain cancers such as cervical, bladder and renal carcinoma secrete Factor H like substances. Factor H binding with pathogens interferes with C3 convertase flagging of pathogens and it destabilizes the function of C3 convertase. This allows pathogens to escape immune surveillance mechanism of host and multiply within host. With reference to HIV, where is the binding site for Factor H? This is detailed on its surface protein gp120/41 is studied by Austrian group of scientists from Innsbruck in collaboration with US based scientists from Utah University. Thus Stoiber H. et al reported in “Interaction of several complement proteins with gp 120 and gp 41, the two envelope glycoproteins of HIV-1” in AIDS, 1995; 9: 19-26 and by Stoiber et al in “Human Complement Proteins C3b, C4b, Factor H and Properdin React with specific sites in gp 120 and gp 41, the Envelope Proteins of HIV-1” in inmunobiology, 1995; 193: 98-113. What is the efficiency of Factor H removal in clearing HIV? This question is methodically researched and has been demonstrated. Stoiber et al in “Efficient Destruction of Human Immunodeficiency Virus in Human serum by inhibiting the protective Action of Complement Factor H and Decay Accelerating Factor (DAF, CD 55), In J. of Exp. Med., 1996; 183: 307-310, explains how and why Factor H depletion lead to efficient HIV clearance. The therapeutic significance of this finding is detailed by Dierich et al from Innsbruck, Austria explains how Factor H removal can be used as “A Complement-ary AIDS Vaccine” in Nature Medicine, 1996; 2: 153-155 as well as by Strobier et al in Immunological Reviews, 2001. Factor H on the surface of HIV inactivates C3 convertase and generates inactive C3 complement fragments. These fragments are used to opsonize HIV particles and gain entry inside CD 4 cells. Speth et al from Innsbruck, Austria detail this aspect in “complement receptors in HIV infection” in Immunological Reviews, 1997; 159: 49-67.

PSS has been studied in the basic science literature for immune modulation involving a. Factor D, b. Factor H, and c. CD 4 components of the immunity. It is also studied for its anti-HIV effects. It is also studied for generating vaccine responses.

A. Factor D Inhibition: The role of PSS for complement inhibition has been studied. U.S. Pat. No. 4,265,908 to Conrow et al describes the complement inhibitory effects of sulfonic salts and its various formulations. The contents of U.S. Pat. No. 4,265,908 is incorporated herein by reference thereto. Several investigators have reported the complement inhibitory properties of PSS. This knowledge has been applied successfully to develop Polysulfone and biosulfane hollow fibers for the therapy of hemodialysis. The successful application of this advances has been reported by Belgium group of scientists (Vanholder et al “Clinical experience with polysulfone: 10 years, in clinical Nephrology, 1994; 42: S13-S20). Successful cure of diabetes and Parkinson's disease by cellular xenotransplantations have also been reported by reputed Japanese group of scientists from Kyoto university (a. Setoyama H. et al “The potential of anticomplement synthetic sulfonic polymers for xenotransplantation, in Transplantation proceedings, 1998; 30: 67-70, b. Iwata H. et al “Control of complement activities for immunoisolation, in Annals of the New York Academy of Sciences, 1999; 875: 7-23 and c. Date I. Et al in “Preliminary report of polymer-encapsulated dopamine secreting cell graft into brain, in Cell Transplantation, 1996; 5: S17-S19). Kirschfink M. summarizes the state of current drugs that are being researched for “Targeting complement in therapy” in Immunological reviews, 2001; 180: 177-189. Geneva and French based group of scientists such as Pascual et al in “Specific interactions of polystyrene biomaterials with factor D of human complement” studied the mechanism of complement inhibition and reported their findings in Biomaterials, 1993; 14: 665-670. PSS inhibits Factor D that is a complement activating protein of alternate complement pathway. Recent advances of immunology have widened the role and functions of Factor D. Factor D is a serine protease of chymotrypsin family. This family of proteins is involved in the complement pathways involving classical, lectin based and alternate complement system as well as in coagulation cascade. Factor D inhibitors therefore are not specific for Factor D alone but they cross inhibit classical, lectin based complement pathways and coagulation pathways as shown by USA based group of scientists (Kilpatrick J. H. et al “Chapter 13: Control of the Alternate Complement Pathway: Inhibition of Factor D in Controlling the Complement system for novel drug development”, 1997:203-225, edited by Mazarakis H and Swart S. J., Published by International Business Communications, Inc. and Rustagi P. K. et al “Development of Novel, Broad spectrum Serine Protease Inhibitors for use as anticoagulants, Chapter 15, Anticoagulant, Antithrombotic and Thrombolytic therapeutics II, 1998, IBC Inc, 307-320). Factor D inhibitors are there for serine protease inhibitors.

B. Factor H inhibition: A curious observation made by Pascual et al in 1993 was the conflicting finding that the drug PSS binds to Factor H twice stronger than Factor D. What is the mechanism and kinetics of Factor H inactivation by PSS? Pascual et al, reports the details. At the time these finding were published, the importance of Factor H in immune evasion was not known. This advances came later on particularly with reference to HIV. Thus finding that Factor H is inhibited strongly by PSS is a predated medical advance ahead of recent advances of immunology. The immense significance of this finding remains to be explored.

C. CD 4 Inhibition: CD 4 receptors are present on monocytes, macrophages and Antigen Presenting Cells or Dendrocytes of innate immune cells and also on CD 4T cells of adaptive immune cells. These receptors work in conjunction with Chemokine receptors such as CCCR5 and CXCR4 to facilitate HIV entry in CD 4 cells. Complement receptors CR1-CR3 and other receptors such as FcyR may also co-participate in this process. Such intact entry of HIV inside CD 4 cells lead to viral multiplication and budding out of fresh viruses. This is detailed by Chaplin J. W. “HIV Pathogenesis: gp 120-antibody complexes bind CD 4 and kill T4 cells-immunotoxin therapy should prevent the progression of HIV to AIDS in Medical Hypotheses, 1999, S2/2: 133-146 and Hewson T. et al “Review Article: Interactions of HIV-1 with Antigen Presenting Cells, in Immunology and Cell biology, 1999, 77: 289-303. What is the role of PSS in inhibiting the surface attachment of HIV? This has been studied independently by Australian group of scientists. Parish et al in “The Journal of Immunology, 1990; 145: 1188-1199.

CD 4 cells of innate immunity are involved in processing foreign antigen peptides and lodging them in the grooves of MHC 1 and MHC 2. This provides secondary non-self identity along with expression of co receptor function such as B7 and adhesion receptors. Dendritic cells essentially act as a transducer between innate and adaptive immunity. CD 4 T and CD 8 T cell initiates adaptive immune responses of vaccine type only if both signals are adequately expressed in CD 4 cells of innate immunity. Failure to express signal 1 and 2 by either drugs or by interference with intracellular processing or destruction of cells by viruses lead to tolerance phenomenon. This prevents the generation of appropriate vaccine responses. This aspect of the immunology is detailed in by a. Bell D. et al “Dendritic cells” Advances in Immunology, 1999; 72, 255-324, and by b. Sousa C. R. et al in “The role of dendritic cells in the induction and regulation of immunity to microbial infection” Current Opinions in Immunology, 1999; 11: 392-399. The recent advances of immunology, thus identifies, CD 4 cells as being present in both innate cells (monocytes, macrophages and dendritic cells) as well as in adaptive cells—CD 4 T cell. HIV primarily uses CD 4 receptors and the associated co receptors to gain entry inside immune cells. This is the primary mechanism used by HIV to defeat the expression of secondary non-self-recognition and its associated memory functions as per two signal hypotheses.

PSS is a drug that stains strongly with Schiff stain (Chaplin A. J. from UK reports “The use of histological techniques for the demonstration of ion exchange resins”, in J. Clin. Pathology, 1999; 52: 776-779. Schiff positive drug reacts with CD 4 T cells (Th0) and stimulates Th1 type cytotoxic adaptive immune responses as well as TH2 type neutralizing Ab type B cell responses. (Rhodes J. et al from Welcome research group in UK reported “Therapeutic potentiation of the immune system by co stimulatory Schiff-base-forming drug, in Nature, 1995; 377: 71-75) and Higaki M. et al from Japan reported “Enhancement of immune response to intranasal influenza HA vaccine by micro particle resin, Vaccine, 1998; 16: 741-745. Pinto L. et al from National Institute of health, USA published “Short Analytical Review: Immune based Approaches for control of HIV infection and viral induced immunopathogenesis” in Clinical Immunology, 2000; 97: 1-8. In developing immune based therapeutic approaches they commented on the benefit of Flu vaccine having anti-HIV like cytotoxic responses.

PSS has been shown to bind to V3 loop of gp 120, inactivate Reverse transcriptase and Tat enzyme in in-vitro and has been experimented for its potential to provide STD prophylaxis by US group of scientists (a. Lee J. J. in U.S. Pat. No. 5,308,612 titled “Uses of polystyrene sulfonate and related compounds as inhibitors of Tran activating transcription factor (TAT) and therapeutics for HIV infection and AIDS; the contents of U.S. Pat. No. 5,308,612 is incorporated herein by reference thereto; b. Mohan et al in “Sulfonic polymers as a new class of human immunodeficiency virus inhibitors” in antiviral Research, 1992; 18: 139-150, c. Tan G. T. in “Sulfonic acid polymers are potent inhibitors of HIV-1 induced cytopathogenicity and the reverse transcriptases of both HIV-1 and HIV-2” Biochemica et Biophysica ACTA, 1993; 1181: 182-188;d. Anderson R. A. et al in “Evaluation of Poly (Styrene-4-Sulfonate) as a Preventive Agent for conception and Sexually Transmitted Diseases” in J. of Andrology, 2000; 21: 862-875 and e. Harold B. et al in “Poly (Sodium 4-Styrene Sulfonate): An Effective Candidate Topical Antimicrobial for the prevention of Sexually Transmitted Diseases, The J. of Infectious Diseases, 2000; 181: 770-773). Careful perusal of HIV immunopathogenesis suggests that these actions are not only anti-HIV effect-it essentially targets HIV's ability to replicate, mutate and induce immunosuppression in the host (a. Foucher R. A. M. et al from Amsterdam, The Nedtherlands reported in “Phenotype-Associated Sequence Variation in the Third Variable Domain of the Human Immunodeficiency Virus Type 1 gp 120 molecule, in J. of Virology, 1992; 3183-3187; b. Zocchi M. R. et al Italian group of scientists studied “HIV-1 Tat inhibits Human Natural Killer Cell Function by Blocking L-Type Calcium Channels, in The J. of Immunology, 1998; 161: 2938-2943); c. Cianciaolo G. J. “Chapter 3: Viral related Proteins in Immune Dysfunction Associated with AIDS” in Human Retroviral infections: Immunological and therapeutic control, edited by Ugen K. E., Bendinelli M. and Friedman H., Kluwer Academic/Plenum Publishers).

In the last decade our knowledge of complement system has expanded as shown in the articles of a. Sahu A. and Lambris J. “Structure and biology of complement protein C3, a connecting link between innate and acquired immunity”, Immunological Reviews, 2001; 180: 35-48. b. Song W. C. et al in “Complement and innate immunity” in Immunopharmacology, 2000; 49: 187-198. The various pathways and complex interrelationship of the working of the immune system is provided by Chapter 19, Antimicrobial Defense System in “Biochemical Pathways: An Atlas of Biochemistry and Molecular Biology” edited by Michael G., Willey J. publication. Complement system is the ancient anti-microbial system that is also involved in xenotransplants and to a subtle extent in allotransplants. There are several complement-mediated diseases for which there is no effective therapeutic drug. Deficiency of complement proteins and failure to generate antibody responses or lytic responses lead to fulminant, life threatening infections. Similarly, piracy of specific complement proteins and/or its receptor functions by certain bacterias, viruses, parasites and cancers lead to immune evasion and opportunistic infections (Wurzner R. in Mini Review: Evasion of pathogens by avoiding recognition or eradication by complement, in part by molecular mimicry, in Molecular Immunology, 1999; 36: 249-260). Complement system and its components have been given new roles of monitoring self/non-self, scavenger function to remove apoptotic cells and immune complexes. The complement system is further involved in initiation of adaptive immunity and memory responses. Therapeutic impacts of these advances are yet to be realized. It has been recently hypothesized that “the simple antigen migration-localization principle should further our understanding of the events that occur with or without therapeutic intervention in a variety of infectious, neoplastic and autoimmune diseases or after transplantation, and may offer improved rationales for prevention and treatment”. (Startzyl T. E. and Zinkernagel R. M., “Review Article: Antigen localization and Migration in Immunity and Tolerance”, The New England Journal of Medicines, Vol. 339, No. 26, pp. 1905-1913).

### SUMMARY OF THE INVENTION

It is the object of the invention to provide cost effective, expedient life saving broad-spectrum immune modulation drug such as PSS to address the global needs of HIV/AIDS crisis.

I achieve above object of the invention by due diligent, careful perusal of the prior art literature and discovered the following

a. Polystyrene sulfonate is a new class of broad-spectrum immune modulatory drug that modulate the humeral and cellular functions of both innate and adaptive immune system. The action of PSS on Factor D, Factor H and CD 4 are the predated, serendipitous advances of immunology whose importance is obvious only after advances in immunology are further defined.

b. When the reported actions of PSS are vertically integrated-PSS—As per recent advances of immunology provides complement inhibition, modulate the initiation of adaptive immune responses and can generate effective Th1 and Th2 type vaccine immune responses.

c. In HIV/AIDS the drug provides “Ultra-broad Spectrum Anti-HIV” activity and immune based therapy. It has therapeutic potential to replace current cumbersome “HAART” therapy with better safety profile.

d. In HIV/AIDS the drug has potential to cover all aspects of HIV prophylaxis including Vaccine therapy.

The role of translational research and recent advances of immunology is emphasized in above conclusions.

My ability to solve above problems resides in the facts that from Bench to bedside, from Concept to Clinical application, and from Discovery to dissemination, translating novel scientific insights into new approaches for prevention, diagnosis, and treatment of disease is the ultimate goal of medical research. Advances in medical research results from a series of interrelated and interdependent steps involving basic scientists, applied researchers and clinical investigators. This translational research process includes verification of basic hypotheses and observation with in vitro studies, confirmation in animal experiments and perhaps refinement with biomathametical modeling, and clinical testing in phase 1 to phase three trials (Fontanrosa P. B. and DeAngelis C. D. in “Editorial: Basic Science and Translational research, JAMA, 2001; 285: 2246). Integration of basic science data from diverse global sources conducted by diverse leading scientists with different purposes, in different time zones to solve critical therapeutic problems of our time is a daunting task and requires sharp analytical skills and deep understanding of the medical sciences. Such analysis if done due diligently saves billions in unnecessary experiments and redirects scarce resources to where it is needed most. In recent times the use of Internet and information processing tools have eased these tasks.

a. PSS is an endocrine drug used by me for therapy of electrolyte disturbances such as hyperkalemia since 1975 (a. Gerstman B. B. and Platt R. “Use of Sodium Polystyrene sulfonate in Sorbitol in the United States, 1985-89 in American J. of Kidney Diseases, 1991; 18: 619-620 and b. Sodium Polystyrene sulfonate, Martindale: The Complete Drug Reference, 32^{nd} edition, edited by Parfitt K., Pharmaceutical Press, 1999; 995-996). Ideas and observations from the clinic can be brought to the laboratory or to a more basic level of the translational research hierarchy for further investigation as shown by the new application of this established drug.

b. Drug discovery: This is often a freelance activity pursued at prestigious medical institutions around the world by scientists of repute. This process does not require regulatory approval. PSS is a globally approved drug that is available in USA since 1975. This drug has several multifaceted properties and therefore a ready source material for researchers around the world. Several researchers around the world have used this material to pursue their research interests in different medical disciplines. Physicians have responsibility to remain aware of current advances in biomedical sciences and to understand the application of promising new ideas to clinical medicine. Several immune modulatory actions are reported with PSS but its integration or translation with current advances of immunology was an overlooked component. This has led to underestimating and overlooking of the true significance of PSS. Our due diligent review identifies PSS as a new class of broad-spectrum immune modulating drug that has capacity to modulate humeral and cellular arms of innate and adaptive immune responses.

c. Drug development process: This is a highly restricted, regulated process that requires addressing safety concerns and efficacy concerns.

Typically, drug regulators require drug sponsor, institutional review board, and clinical trial data in a systemic manner from phase 1 to phase 3 and phase 4 post marketing monitoring of the drug. This is a multimillion dollars undertaking requiring extensive documentations. Only drug industry is able to undertake or afford such activity. Government institutions and medical scientific establishments in general undertake clinical trials sponsored by drug companies. Their own ability to initiate drug trials is severely handicapped due to regulatory burden and costs. In certain disease conditions such as immune diseases surrogate markers are used extensively to judge the efficacy of the drug. Here independent efficacy data by at least two sources provide considerable validity. New applications of established drug is an exception to the drug development process. It provides safety data that normally cost millions of dollars to evaluate prospectively. It provides global availability and ready formulations that require minor modifications. In a deadly disease such as HIV/AIDS where current therapeutic measures fail—use of new untried formulations can be justified in current cases where “HAART” therapy fail. It provides an opportunity to save lives and provide rapid meaningful data that can be improvised for lesser urgent medical conditions. In developing several therapeutic applications related to immune diseases, it might be noted that careful perusal of the data show that clinically approved dosages of the drug are on the average 90 gram/day for hyperkalemia therapy. At these doses, the safety of the drug is evaluated for mucosal applications (oral and rectal) in millions of patients. The dose required for immune applications is less than 1 gram per day as shown by in-vitro and in-vivo results of the drug. This information is directly transferable to therapeutic applications and require minimal burden of proof. Global resources if directed to further develop this drug will provide the most efficient solution to current “Immune related Global Problems”.

### BRIEF DESCRIPTION OF THE DRAWINGS

In the accompanying drawings to which reference is made in the instant specifications which is to be read in conjunction therewith, like reference numerals are used to indicate the parts in the various views:

FIG. 1 is a flowchart illustrating the Alternate Complement System and its vertical integration with recent advances of immunology;

FIG. 2 is a flowchart illustrating the short-circuiting of immune responses by HIV and its piracy of Factor H; and

FIG. 3 illustrates the life cycle of HIV in the host and how PSS provides ultra-broad spectrum anti-HIV effects and immune-modulation.

### DESCRIPTION OF THE PREFERRED EMBODIMENT

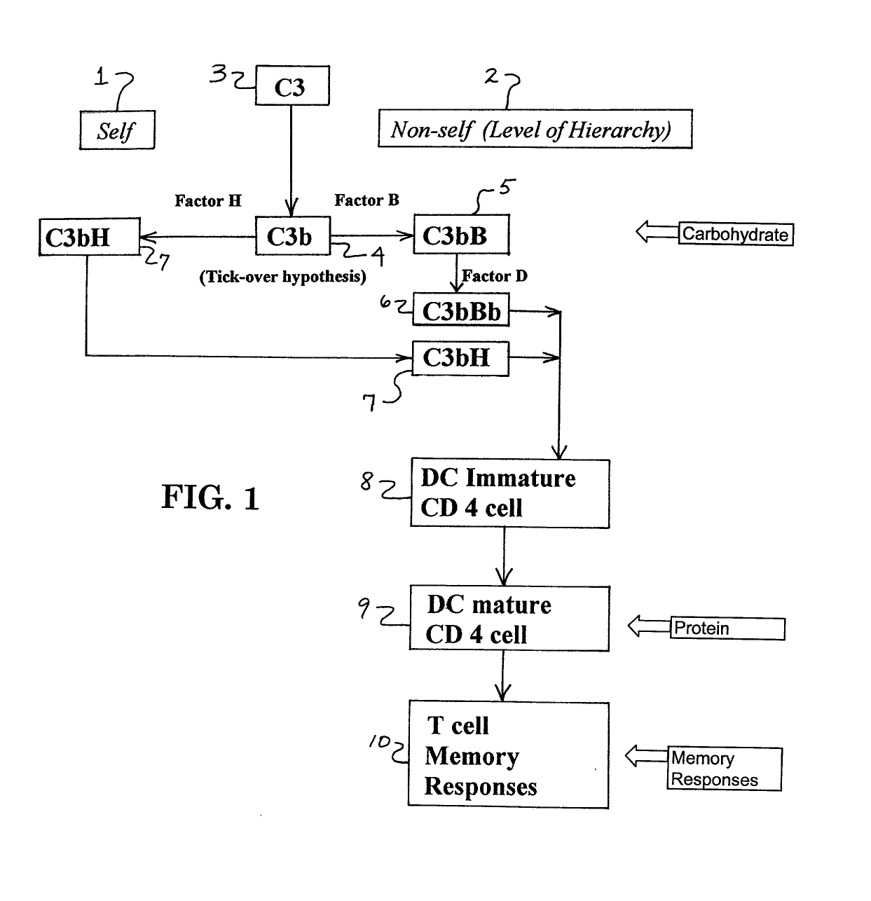

As shown in the FIG. 1, the innate complement system differentiates self, 1 and non-self, 2 through C3, 3 and its active catalysed product, C3b, 4. Identification of polyanionic surface on host surface by Factor H, lead to formation of C3bH, 7. In the absence of Non-self no further C3 convertase is formed. C3b is constantly generated due to “enzyme tick-over hypothesis”. If Non-self or bacteria is identified because of carbohydrate signature it carries, C3b, 4 bind preferably to Factor B to generate C3bB, 5. This is further acted upon by Factor D to generate C3 convertase, C3bBb, 6. C3bH, 7 now has a regulatory function. Active C3 fragmented products, C3a-C5a and C5b-C9, membrane attack complex have inflammatory components. C5b-C9 in addition has cytotoxic component that form membrane attack complex and cause cytolysis. The active C3 fragments contribute to CD 4 or dendritic cell, 8 by expression of co receptor function such as B 7 molecule. Inactive C3 fragments generated by C3bH, 7 contribute to CD 4, dendritic cell, 8 for phagocytosis response of fragmented antigens. CD 4, dendritic cell, 8 processes antigen fragments inside cell and lodge them in the groove of MHC 1 and MHC 11. This allows CD 4 cells, dendritic cell, 9 to mature. T cell, 10 identify secondary Non-self identity in accordance to “two signal hypothesis”. This means, both signal 1 of MHC 1 and 11 and signal 2 of B7 co receptor function must be expressed. If they are not expressed, CD 4 T cells, will not sense the presence of Non-self and no clonal or memory responses are generated.

Human defense system is much like military defense organization of the country by analogy. Factor H is the controlling element that acts as radar and scans for self and determines the primary identity of self. This is done by identifying polyanionic or sialic acid binding sites as self. Factor D identifies non-self. The primary identity of non-self by the innate immunity depends upon its ability to recognize carbohydrate signature. Factor D (25 KD) is the complement protein of the alternate defense system that has the smallest molecular weight. It is ubiquitously present through out the body and therefore a convenient molecule to sense the non-self as primary danger signal. Upon encountering non-self, it triggers a chemical alarm system in the form of activation and amplification of the alternate complement system. The function of activated (C3a, C4a and C5a) and amplified alternate complement system (C5b-C9) is to localize the non-self antigen, produce inflammatory responses around it, mobilize the army of leucocytes and CD 4 containing cells (monocytes, macrophages and dendritic cells) and remove the non-self antigen expediently. Factor H besides identifying self controls the degree of alarm function by generating inactive C3 fragments. C3 convertase along with inactive C3 fragments flag the nonself; opsonize it for phagocytosis by CD 4 cells. CD 4 cells that are involved in the phagocytosis and expression of secondary Non-self expression are immature dendritic cells. Inactive C3 fragments are also involve in stimulating B cell function and act as its co-receptors. CD 4 cells process the non-self antigen inside its cellular machinery and flag its peptide fragments in MHC 1 and MHC 2 groove. The active C3 fragments also influence CD 4 cells to express co receptor such as B7 and adhesion receptors. Once secondary expression of Non-self is complete, CD 4 cells are now called as matured dendritic cells. CD 4 T cells and CD 8 T cells sense the presence of non-self peptide fragments only in the presence of associated co receptors such as B7. Thus expression of two foreign signals in denditic cell provides the secondary identity of non-self. T cells sense the secondary non-identity of dendritic cell only if two signals are expressed. The identity of self is maintained if dendritic cells are not matured as in normal state, or if dendritic cell maturation of two signals is prevented or subverted either by drugs or by pathogens such as viruses. The secondary identity of non-self provides back up responses in the form of clonal expansion and mobilization of T cell responses in the form of neutralizing Ab and cytotoxic cells to further assist human defense system for the removal of non-self. Co receptor function imparts memory function to T cells. In the case of repeat invasion by non-self, back up reserve force is directly activated to deal with the intrusions. The vertical integration of Factor H, D and CD 4 function in FIG. 1 is thus in accordance to recent advances of immunology where Non-self identity has three levels of hierarchy (1-3). Each level must proceed sequentially to be fully effective.

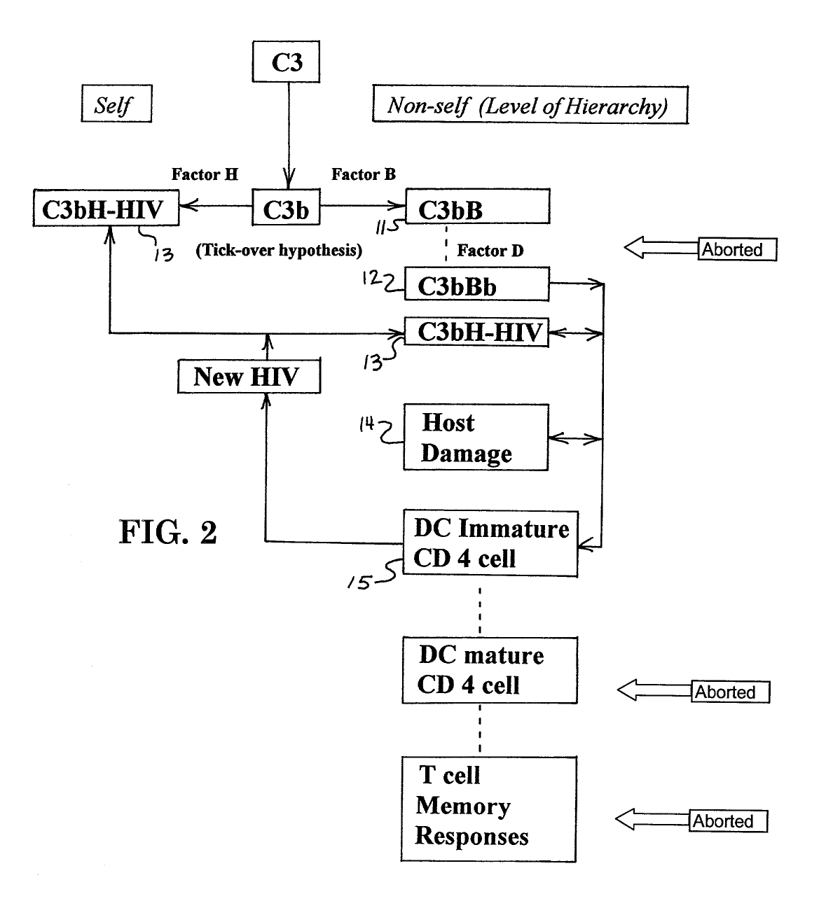

As shown in the FIG. 2, HIV as an intruder to human defense system encounters complement system. It is an enveloped virus and has allo MHC 1 and MHC 2 proteins of the primary host and its complement regulatory machinery on its surface. However, it is sensed efficiently by the complement system as an intruder and alarm system is triggered. HIV simultaneously binds to host Factor H to form C3bH-HIV, 13. This is similar to hijacking of the controlling element of the defense system. Recruited CD 4 cells at inflammatory sites no longer can identify or locate the intruder or non-self. HIV thus masks the primary identity of non-self. C3 flagging of non-self HIV do not occur and complement system though activated (C3a, C4a and C5a) it does not proceed to C5b-C9 to generate cytotoxic responses (1-aborted). HIV by hyper-activating alarm system, C3bBb, 12 inflicts inflammatory damage to the host, 14. Activated C3 fragments are now inactivated by Factor H hijacked by HIV. Inactive C3 fragments are used to gain entry inside CD 4 cells, 15. Human defense system fails to identify intruder as non-self, it fails to flag intruder and fails to efficiently fragment HIV. HIV now exploits complement fragments and receptor machinery of cells for successful entry inside CD 4 cells or immatured dendritic cell, 15. The very cells that suppose to phagocytose HIV and present its peptide fragments to MHC 1 and 2 now supports the multiplication of HIV. This aborts secondary Non-Self identity (2-aborted), 15. HIV multiplication generates HIV proteins that further cause immunosuppression and aborts expression of memory responses. HIV thus masks the secondary identity of non-self by preventing dendritic cell maturation and subverting its function for its own survival and replication. Masking of secondary identity of non-self prevents generation of secondary defense forces in the form of neutralizing Ab and cytotoxic cells. The New HIV budding out and repeats the cycle of CD 4 entry to systemically destroy its immune function and pave the way for AIDS by short-circuiting the functioning of the human defense system, 16.

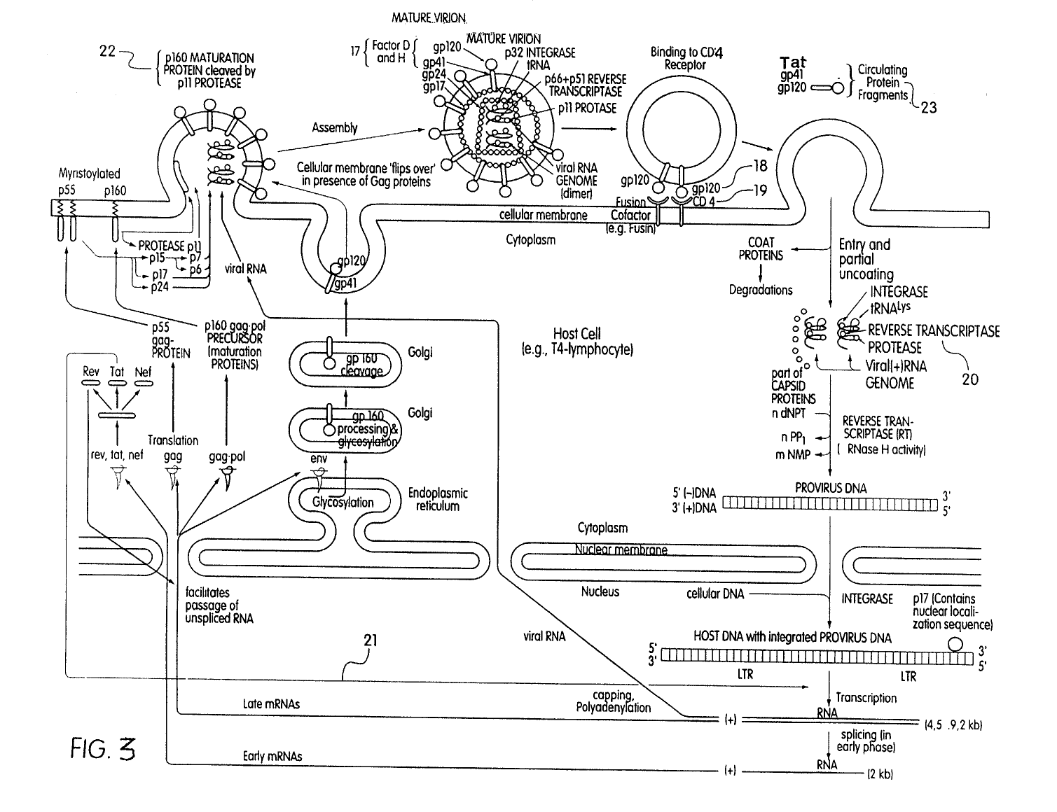

As shown in FIG. 3, PSS is a polyanionic, foreign biomaterial that mimics the sialic acid or polyanionic properties of host cells. It competes with HIV for Factor H, 17, Factor D, 17 and CD 4 cell surface, 18-19. This results in inhibition of Factor D, Factor H and surface attachment of HIV to CD 4 cells, 17-19. In addition, HIV proteins such as reverse transcriptase (RT), 20 is inhibited outside the cells. Similarly, Tat protein, 21 is inhibited. Serine proteases are involved in gp120 maturation. PSS, being a serine protease inhibitor is expected to inhibit 22. HIV proteins are inhibited mainly outside cells, 23 that are involved in immunosuppression Thus PSS is a new class of broad immune modulation drug that can affect the humeral and cellular function of innate and adaptive immune system and provide ultra-broad spectrum anti-HIV effects.

Table 1, details the action of PSS and its intended immune modifying effects on host as well as HIV.

TABLE 1
PSS effects: Therapeutic Immune responses

| NO. | Immune component | PSS Actions | Effects | |
|---|---|---|---|---|
| 1 | Innate (Humeral) | Inhibition *2 | 1. | HIV exposed to circulating Ab |
| | Factor H | | 2. | HIV killed efficiently by circulating Ab |
| | | | | HIV fragments processed by CD 4 cells for |
| | | | | secondary level of Non-self hierarchy. |
| 2 | Innate (Humeral) | Inhibition *1 | 1. | Reduce host inflammation |
| | Factor D | | 2. | Pathway proceed to C5b-C9 level |
| | Innate and Adaptive | Surface inhibition | 1. | Reduce CD 4 entry |
| | (cellular) | | 2. | Reduce CD 4 replication |
| | CD 4 (Dendritic cells, | | 3. | Reduce CD 4 HIV proteins secretions |
| | Immature) | | 4. | Reduce immunosuppression |
| | | | 5. | Reduce CD 4 destruction |
| 4 | HIV proteins | Inhibition | 1. | Reduce Replication (RT, Tat) |
| | (V3 loop of gp 120, | | 2. | Reduce Mutational change (V3, RT) |
| | Reverse Transcriptase | | 3. | Reduce immunosuppression (gp120, Tat) |
| | (RT), Tat | | | |
| 5. | Innate and adaptive | Stimulated | a. | Neutralizing Ab responses |
| | immune cells | i. Adjuvant | b. | Cytotoxic responses |
| | CD 4-CD 4 T cells | effect for Th2 | | (Vaccine like effects) |
| | Interactions | ii. Schiff positive | | |
| | | Th1 responses. | | |

My U.S. Pat. No. 5,976,780 titled “Encapsulated Cell Device” details the formulation strategy to combine PSS with ultra-purified biomaterial that reduce nonspecific or background immune activation and meets the endotoxin content criteria's as recommended by FDA. The contents of U.S. Pat. No. 5,976,780 is incorporated herein by reference thereto. The immunoisolation strategy allows selective control of pore size to regulate the entry of desired immune proteins and can be used to develop drug devices to meet disease specific requirements for blood contacting applications.

What is claimed is:

1. A method of treating a mammal in need of immune modulation comprising the step of:
administering to the mammal an effective amount of polystyrene sulfonate as a broad spectrum immune modulatory compound to modulate the humeral and cellular arms of innate and adaptive immunity by modulation of a compound selected from the group consisting of Factor H, Factor D, CD4 cells, and combinations thereof.

2. The method of claim 1, wherein the immune modulatory compound effects HV interaction with Factor D and Factor H.

3. The method of claim 2, wherein the immune modulatory compound displaces HIV surface attachment to CD4 cells.

4. The method of claim 3, wherein the immune modulatory compound reduces HIV inhibition of Factor D and Factor H.

5. The method of claim 4, wherein the immune modulatory compound inhibits HIV proteins.

6. The method of claim 5, wherein the HIV protein comprises reverse transcriptase.

7. The method of claim 5, wherein the HIV protein comprises Tat protein.
